(19) United States
(12) Patent Application Publication
(10) Pub. No.: US 2013/0245663 A1
(43) Pub. Date: Sep. 19, 2013

(54) Devices, Systems and Methods for Treatment of Eating Disorders

(71) Applicant: PLENSAT, INC., Oakland, CA (US)

(72) Inventors: Houston Fredrick Voss, San Clemente, CA (US); Bernhard B. Sterling, Danville, CA (US)

(73) Assignee: Plensat, Inc., Oakland, CA (US)

(21) Appl. No.: 13/875,086

(22) Filed: May 1, 2013

Related U.S. Application Data
(63) Continuation of application No. 12/930,377, filed on Jan. 4, 2011, now abandoned.
Application No. 12/930,377 is a continuation-in-part of application No. 11/270,273, filed on Nov. 9, 2005, now Pat. No. 7,686,892.
Application No. 11/270,273 is a continuation of application No. 11/270,397, filed on Nov. 8, 2005, now Pat. No. 7,674,396.

(51) Int. Cl. A61F 5/00

(52) U.S. Cl. CPC A61F 5/003
USPC 606/192

(57) ABSTRACT

An ingestible gastric device comprising a formed body that is adapted to expand upon exposure to a hydration medium and then degrade after a first residence time in the stomach cavity, allowing the device to be passed by the patient's normal digestive process, and monitoring means for monitoring the gastric device. In some embodiments, the monitoring means comprises a biocompatible RFID tag.

### CROSS-REFERENCE TO RELATED APPLICATIONS

This application is a continuation-in-part of U.S. application Ser. No. 11/270,273, filed Nov. 8, 2005, which is a continuation of U.S. application Ser. No. 11/270,397, filed Nov. 8, 2005, now U.S. Pat. No. 7,674,396.

### FIELD OF THE PRESENT INVENTION

The present invention relates generally to the treatment of eating and gastrointestinal disorders. More particularly, the invention relates to expandable gastric devices that can reside in the stomach for a controlled and prolonged period of time, and methods and systems for monitoring the gastric devices.

### BACKGROUND OF THE INVENTION

According to the American Medical Association, obesity is reaching epidemic proportions, affecting over 30% of American adults, or almost 70 million people. The percentage of affected adults (and children) is also climbing. In addition to the health risks presented by obesity itself, obesity increases the likelihood of a wide range of significant co-morbid health risks including cardiovascular complications (such as hypertension and hyperlipidemia), diabetes, gallbladder disease, cancer, polycystic ovary disease, pregnancy-related problems, arthritis-related problems and other orthopedic complications caused by stress on body joints.

Obesity can have several causes. Genetic, environmental and psychological factors are all believed to play a role in obesity. The mechanism for weight gain includes impaired metabolism of adipose tissue, physical inactivity (due to lifestyle or other illness), and uncontrolled appetite. Some illnesses, such as hypothyroidism, Cushing's disease and depression can also lead to obesity partly through hormonal effects, and partly through changes in appetite and lifestyle.

Regarding hormonal effects on obesity, the control of thyroid hormone secretion and adrenal gland secretion is at the level of the hypothalamus and pituitary regions of the brain. The hypothalamus secretes thyroid releasing factor which leads to release of thyroid stimulation hormone from the pituitary gland leading to increases in thyroid hormone production and release from the thyroid gland. In a similar fashion Corticotrophin releasing factor released from the hypothalamus leads to release of adrenocorticotrophic hormone that causes increased cortisol secretion from the adrenal glands causing Cushing disease.

Obesity can further be caused by certain drugs, such as steroids and some antidepressants. These effects are also thought to occur in the appetite centers in the brain.

Obesity is a common feature of neurologic diseases that appear to affect the appetite control center in the hypothalamic, pituitary and brain stem regions of the brain. Kline-Levine syndrome and sarcoidosis of the hypothalamus, for example, are associated with massive obesity.

When diet therapy proves ineffective, morbid obesity is often treated through bariatric surgery. Common bariatric surgical procedures include adjustable gastric banding and vertical banded gastroplasty (VBG).

In the noted surgical procedures, a band is surgically placed around the upper part of the stomach creating a small pouch. The pouch fills quickly when eating or drinking giving the patient the sensation of satiety.

Another popular treatment is the Roux-en-Y gastric bypass, in which a small stomach pouch is created and a section of the small intestine is attached to the pouch to allow food to bypass the lower stomach, the duodenum, and the first portion of the jejunum. This bypass reduces the amount of calories and nutrients the body absorbs.

It is also known to surgically insert a gastric balloon into the stomach to assume a portion of the volume of the stomach cavity, and, hence, reduce the available capacity of the stomach for food. This has the effect of reducing appetite and, consequently, over time (e.g., 3 months), and in combination with a suitable diet, causes weight loss.

Intragastric balloons, such as the device disclosed in U.S. Pat. No. 5,084,061, are typically designed to provide short-term therapy for moderately obese individuals that require a reduction in weight prior to surgery, or as part of a dietary or behavioral modification program. Such devices are typically inserted into the stomach cavity in an outpatient setting (i.e., under endoscopic control), using local anesthesia and sedation.

After placement, the balloons are filled with saline solution or air from outside the cavity. Placement is typically for a period from 6 to 12 months. Removal of the balloons generally requires endoscopy.

A number of gastric balloon systems have also been employed that permit the volume of a gastric balloon to be varied over time. The purpose of facilitating volumetric changes in the gastric balloon is to provide periods of feelings of relief and well-being to the patient; not to prevent balloon accommodation and maintain appetite reduction. One such system is disclosed in U.S. Pat. No. 4,133,315, which utilizes a flexible filling/release tube permanently coupled to the balloon. An even more invasive, surgically implanted tube design is described in U.S. Pat. No. 5,234,454 to Bangs.

In U.S. Pat. No. 5,084,061, a free floating gastric balloon is disclosed that includes a valve that can be detachable coupled to a filling tube. The device requires a physician's care and sedation for adjustment.

In addition to undesirable system complexity, each of the above-described bariatric procedures has associated risks. A significant concern with banded surgeries is a high incidence of complications, such as bleeding and/or obstruction. Though generally better tolerated than banded procedures, the Roux-en-Y gastric bypass still results in significant complications, such as vitamin and mineral deficiencies, and may lead to osteoporosis in the long-term.

Additionally, while any surgical procedure involves risks, surgical procedures on obese patients present significantly higher risks of complications and death. The obesity makes it difficult to administer anesthesia in proper doses. The surgical wounds often do not heal properly. Obese patients also face a higher risk of complications after surgery, such as deep venous thrombosis.

A further means of treating eating disorders comprises ingesting one or more gastric retention devices that are adapted to reside in the stomach for a controlled and prolonged period of time to reduce the volume of the stomach (or gastric) cavity. Many of the prior art devices are further adapted to expand upon exposure to gastric fluids to further enhance the reduction of gastric volume. Illustrative are the gastric retention devices disclosed in U.S. application Ser. Nos. 10/741,177 (Pub. No. 2004/0192582A1) and 10/778,917 (Pub. No. 2004/0219186).

The gastric retention device disclosed in U.S. application Ser. No. 10/741,177 comprises an acid sensitive, gelatin coating over a dehydrated hydrophilic polymer. When ingested, the acid-sensitive coating is dissolved by gastric secretions and the hydrophilic polymer is exposed to the aqueous environment of the gastric milieu. Upon exposure to the aqueous environment, the hydrophilic polymer expands to reduce the gastric volume.

There are several drawbacks and disadvantages associated with the gastric retention device disclosed in U.S. application Ser. No. 10/741,177. A significant disadvantage is the lack of containment for the polymer, which is capable of swelling by hydration with a gastric fluid.

The gastric retention device disclosed in U.S. application Ser. No. 10/778,917 includes an expandable gel matrix having one or more monomeric or polymeric materials, and at least one diagnostic or therapeutic agent, and/or imaging agent. The gel matrix, when dried, is then inserted into a gastrically erodible capsule.

There are similarly several drawbacks and disadvantages associated with the gastric retention device disclosed in U.S. application Ser. No. 10/778,917. A significant drawback is that the disclosed device is designed and adapted for controlled and predictable delivery of pharmaceutically active agents, which limits the size, thickness and volume of the retention system.

A further disadvantage of the disclosed gastric retention devices is that they do not include any means of tracking or monitoring the devices prior to and/or after ingestion by a subject. Thus, the only means of monitoring an ingested device is through a subsequent medical procedure, such as an xray or ultrasound examination. As will readily be appreciated by one having ordinary skill in the art, such procedures are often time consuming and costly, and require skilled personnel to perform the procedures.

It would thus be desirable to provide low-risk, unobtrusive and noninvasive methods and systems for treatment of eating disorders that readily prevent stomach distention and/or alternatively allow for simple, frequent and timely adjustments of the stomach cavity that is available for food intake.

It would further be desirable to provide ingestible gastric devices and systems for treatment of eating disorders that include means for tracking and/or monitoring the devices prior to and/or after ingestion.

It is therefore an object of the present invention to provide methods and systems for treatment of eating disorders that are low-risk, unobtrusive and noninvasive.

It is another object of the invention to provide readily ingestible (i.e., easy to swallow) gastric devices, which, when expanded, reduce the volume of the stomach cavity and, hence, the amount of food ingested to reach a feeling of fullness.

It is another object of the invention to provide ingestible, inflatable gastric devices having controlled rate of inflation and, hence, expansion.

It is another object of the invention to provide expandable, inflatable gastric devices that are readily degradable (or dissoluble) by gastric fluid and/or intestinal contents in the small and/or large intestines.

It is another object of the invention to provide ingestible, expandable gastric devices having controlled rate of degradation.

It is another object of the invention to provide ingestible, expandable gastric devices having means for tracking and/or monitoring the devices prior to and/or after ingestion.

### SUMMARY OF THE INVENTION

In accordance with the above objects and those that will be mentioned and will become apparent below, in one embodiment of the invention, there are provided ingestible gastric devices comprising (i) a formed body that is adapted to expand upon exposure to a hydration medium, and (ii) monitoring means for monitoring the gastric devices.

In some embodiments, the body includes an outer layer that is adapted to receive a core material therein.

In some embodiments, the monitoring means comprises a passive RFID tag.

In some embodiments, the RFID tag is encapsulated in a biocompatible polymeric material. In some embodiments, the polymeric material is selected from the group consisting of poly(urethanes), poly(siloxanes), poly(methyl methacrylate), poly(vinyl alcohol) for hydrophilicity and strength, poly(ethylene), poly(vinyl pyrrolidone, poly(2-hydroxy ethyl methacrylate), poly(N-vinyl pyrrolidone), poly(methyl methacrylate), poly(vinyl alcohol), poly(acrylic acid), polyacrylamide, poly(ethylene-co-vinyl acetate), poly(ethylene glycol), poly(methacrylic acid), polylactides (PLA), polyglycolides (PGA), poly(lactide-co-glycolides) (PLGA), polyanhydrides, polyorthoesters, like materials and combinations thereof.

In another embodiment of the invention, there are provided ingestible gastric devices comprising flexible, multi-component devices that include a biodegradable outer layer or skin that encloses a sealed cavity (i.e. a pouch). Disposed within the sealed cavity is a hydratable core material that is adapted to effectuate expansion of the cavity and, hence, gastric devices upon exposure to a hydration medium, such as gastric fluid.

In some embodiments of the invention, the outer layer or skin comprises a gel-forming material. In one embodiment of the invention, the gel-forming material is selected from the group consisting of acacia (gum Arabic), agar, agarose, alginate gels, alginic acid, alginate gums, amylopectin, arbinoglactan, carob bean gum, carrageenan, chondroitin sulfate, eucheuma, fucoidan, furcellaran, gelatin, gellan, guar gum, gum ghatti, gum karaya, hypnea, karaya, laminaran, locust bean gum, natural gum, pectins, starches, polypeptides, polyamino acids, tragacanth, xanthan, psyllium, maltodextrin, Carbopol® acidic carboxy polymer, hydrophilic poly urethanes, hydroxypropyl methyl cellulose, HYPOL® hydrophilic polyurethane polymers, polycarbophil, polymethylvinylether co-maleic anhydride, polyvinylpyrrolidone, polyethylene oxide, poly(hydroxyalkyl methacrylate), polymethacrylic acid, poly(electrolyte complexes), poly(vinyl acetate) cross-linked with hydrolysable bonds, polyvinyl alcohol, water-swellable N-vinyl lactams polysaccharides, hydroxypropyl cellulose, carboxylmethyl celluloses, hydroxyethyl cellulose, methyl cellulose, polyvinyl alcohol, hydroxymethyl methacrylate, Cyanmer® polyacrylamides, Good-rite® polyacrylic acid, starch graft copolymers, Aqua-Keeps® acrylate polymer, ester cross linked polyglucan, polycellulosic acid, polyurea, polyether, poly(acrylic acid), polyacrylamide, or poly(2-hydroxyethyl methacrylate, hydroxymethyl methacrylate, methocel, alpha-polyhydroxy acids, polyglycolide (PGA), poly(L-lactide), poly(D,L-lactide), poly(.epsilon.-caprolactone), poly(trimethylene carbonate), poly(ethylene oxide) (PEO), poly(.beta.-hydroxybutyrate) (PHB), poly(.beta.-hydroxyvalerate) (PHVA), poly(p-dioxanone) (PDS), poly(ortho esters), tyrosine-derived polycarbonates, like materials and mixtures thereof.

According to the invention, the skin can also include reinforcing fibers. In some embodiments of the invention, the reinforcing fibers comprise soluble reinforcing fibers, preferably selected from the group consisting of polysaccharide gums, including, without limitation, carboxymethyl cellulose, methocel, carrageenan, guar gum, algimate gels, pectins, xanthan, gum Arabic (acacia), gum tragacanth, karaya, agar, gellan, alpha-polyhydroxy acids, polyglycolide (PGA), poly(L-lactide), poly(D,L-lactide), poly(.epsilon.-caprolactone), poly(trimethylene carbonate), poly(ethylene oxide) (PEO), poly(.beta.hydroxybutyrate) (PHB), poly(.beta.-hydroxyvalerate) (PHVA), poly(p-dioxanone) (PDS), poly(ortho esters), tyrosine-derived polycarbonates, like materials and mixtures thereof.

In some embodiments, the reinforcing fibers comprise insoluble reinforcing fibers, preferably selected from the group consisting of insoluble polysaccharides, cellulose, phenylpropane molecules polymers, lignin, bast fibers, Kozo, Gampi, grasses, kenaf, bagasse, jute, hemp, flax, like materials and mixtures thereof.

In some embodiments of the invention, the outer surface of the skin also includes a hydration modifying material, such as agar, and/or an ingestion enhancing material, such as gelatin.

In some embodiments of the invention, the core material similarly comprises one of the aforementioned gel-forming materials.

In some embodiments of the invention, the core material comprises a gel-forming material selected from the group consisting of acacia (gum Arabic), agar, agarose, alginate gels, alginic acid, alginate gums, amylopectin, arbinoglactan, carob bean gum, carrageenan, chondroitin sulfate, eucheuma, fucoidan, furcellaran, gelatin, gellan, guar gum, gum ghatti, gum karaya, hypnea, karaya, laminaran, locust bean gum, natural gum, pectins, polypeptides, polyamino acids, tragacanth, xanthan, like materials and mixtures thereof.

In some embodiments of the invention, the gel-forming material comprises a gas producing material.

In some embodiments of the invention, the gel-forming material comprises a foam.

In some embodiments of the invention, the gastric devices are configured to hydrate after contact with a hydration medium, such as gastric fluid, within a period of time in the range of approximately 5-30 min. In some embodiments, the gastric devices are configured to hydrate after contact with a hydration medium within a period of time in the range of approximately 5-10 min.

According to the invention, the gastric devices have a first volume prior to exposure to a hydration medium and a second (expanded) volume after substantially complete hydration by a hydration medium that is greater than the first volume; the second volume being in the range of approximately 3-50 times greater than the first volume.

In some embodiments of the invention, the gastric devices are configured to fully hydrate and reach the expanded second volume after contact with a hydration medium for a period of time less than approximately 1 hr.

In some embodiments of the invention, the gastric devices are configured to fully hydrate and reach the expanded second volume after contact with a hydration medium for a period of time in the range of approximately 10-30 min.

In a preferred embodiment of the invention, the gastric devices are configured to degrade after a first period of time to allow the gastric devices to enter a patient's duodenum. In some embodiments of the invention, the first period of time is in the range of approximately 1 hr-1 year. In some embodiments, the first period of time is in the range of approximately 3 hrs-3 weeks.

In some embodiments of the invention, the gastric devices include means for monitoring the devices prior to and after administration or ingestion. In some embodiments, the monitoring means comprises a biocompatible RFID tag.

### BRIEF DESCRIPTION OF THE DRAWINGS

Further features and advantages will become apparent from the following and more particular description of the preferred embodiments of the invention, as illustrated in the accompanying drawings, and in which like referenced characters generally refer to the same parts or elements throughout the views, and in which:

FIG. 1 is an illustration of a portion of the digestive system of a human subject;

FIG. 2 is a partial section view of a portion of the digestive system;

FIG. 3A is a perspective view of one embodiment of a pre-hydration gastric device, according to the invention;

FIG. 3B is a perspective, sectional view of the gastric device shown in FIG. 3A after hydration and expansion, according to one embodiment of the invention;

FIG. 4A is a front plane view of another embodiment of a pre-hydration gastric device, according to the invention;

FIG. 4B is a front plane, sectional view of the gastric device shown in FIG. 4A after hydration and expansion, according to one embodiment of the invention;

FIG. 5 is an illustration of a portion of the digestive system of the subject, showing placement of gastric devices of the invention in the stomach cavity, according to one embodiment of the invention;

FIG. 6A is a perspective view of another embodiment of a pre-hydration gastric device, according to the invention;

FIG. 6B is a side plane, sectional view of the gastric device shown in FIG. 6A, according to one embodiment of the invention;

FIG. 6C is another side plane, sectional view of the gastric device shown in FIG. 6A, after hydration and expansion, according to one embodiment of the invention;

FIG. 6D is a side view of another embodiment of a pre-hydration gastric device, according to the invention;

FIG. 7 is a top plane view of a prior art RFID tag;

FIG. 8 is a front plane view of one embodiment of an encapsulated RFID tag, according to the invention;

FIG. 9 is a front plane view of another embodiment of an encapsulated RFID tag, according to the invention;

FIG. 10 is a partial sectional view of the gastric device shown in FIG. 6B, showing a placement of the RFID tag shown in FIG. 8 within the device skin, according to one embodiment of the invention; and

FIG. 11 is a partial sectional view of the gastric device shown in FIG. 6B, showing a placement of the RFID tag shown in FIG. 8 within the device cavity, according to one embodiment of the invention.

### DETAILED DESCRIPTION OF THE INVENTION

Before describing the present invention in detail, it is to be understood that this invention is not limited to particularly exemplified materials, methods or structures as such may, of course, vary. Thus, although a number of materials and methods similar or equivalent to those described herein can be used in the practice of the present invention, the preferred materials and methods are described herein.

It is also to be understood that the terminology used herein is for the purpose of describing particular embodiments of the invention only and is not intended to be limiting.

Unless defined otherwise, all technical and scientific terms used herein have the same meaning as commonly understood by one having ordinary skill in the art to which the invention pertains.

Further, all publications, patents and patent applications cited herein, whether supra or infra, are hereby incorporated by reference in their entirety.

Finally, as used in this specification and the appended claims, the singular forms “a, “an” and “the” include plural referents unless the content clearly dictates otherwise. Thus, for example, reference to “a gastric device” includes two or more such devices; reference to “core material” includes two or more such materials and the like.

### Definitions

The term “gastric device”, as used herein, means a device having an internal cavity and/or means for forming same (e.g., balloon, pouch, etc.), having a size that is suitable for oral administration to a subject and, following administration, expands to a size greater than the administration size.

The term “gastric fluid”, as used herein, means and includes the endogenous fluid medium (or juices) of the stomach, including secretions therein. The term “gastric fluid” also means and includes ingested fluids, water and simulated gastric fluid.

The term “hydratable”, as used herein, means and includes, the ability of a material to absorb an aqueous material (i.e. hydration medium), such as water, upon exposure thereto (i.e. hydrate) and retain a significant fraction of the hydration medium within its structure thereafter.

The term “gel-forming material”, as used herein, means a hydratable material that preferably expands upon exposure to a hydration medium, including, without limitation, acacia (gum Arabic), agar, agarose, alginate gels, alginic acid, alginate gums, amylopectin, arbinoglactan, carob bean gum, carrageenan, chondroitin sulfate, eucheuma, fucoidan, furcellaran, gelatin, gellan, guar gum, gum ghatti, gum karaya, hypnea, karaya, laminaran, locust bean gum, natural gum, pectins, starches, polypeptides, polyamino acids, tragacanth, xanthan, psyllium, maltodextrin, Carbopol® acidic carboxy polymer, hydrophilic poly urethanes, hydroxypropyl methyl cellulose, HYPOL® hydrophilic polyurethane polymers, polycarbophil, polymethylvinylether co-maleic anhydride, polyvinylpyrrolidone, polyethylene oxide, poly(hydroxyalkyl methacrylate), polymethacrylic acid, poly(electrolyte complexes), poly(vinyl acetate) cross-linked with hydrolysable bonds, polyvinyl alcohol, water-swellable N-vinyl lactams polysaccharides, hydroxypropyl cellulose, carboxylmethyl celluloses, hydroxyethyl cellulose, methyl cellulose, polyvinyl alcohol, hydroxymethyl methacrylate, Cyanmer® polyacrylamides, Good-rite® polyacrylic acid, starch graft copolymers, Aqua-Keeps® acrylate polymer, ester cross linked polyglucan, polycellulosic acid, polyurea, polyether, poly(acrylic acid), polyacrylamide, or poly(2-hydroxyethyl methacrylate, hydroxymethyl methacrylate, methocel, alpha-polyhydroxy acids, polyglycolide (PGA), poly(L-lactide), poly(D,L-lactide), poly(.epsilon.-caprolactone), poly(trimethylene carbonate), poly(ethylene oxide) (PEO), poly(.beta.-hydroxybutyrate) (PHB), poly(.beta.-hydroxyvalerate) (PHVA), poly(p-dioxanone) (PDS), poly(ortho esters), tyrosine-derived polycarbonates, like materials and mixtures thereof.

The term “viscosity building material”, as used herein, means a hydratable material that retains a significant fraction of formed gas upon exposure to a hydration medium. Viscosity building materials thus include, without limitation, gel-forming agents, water soluble polymers, sodium and calcium polyacrylic acid, polyacrylic acid, polymethacrylic acid, polymethylvinylether co-maleic anhydride, polyvinylpyrrolidone, polyethylene oxide, hydroxypropyl cellulose, hydroxypropyl methyl cellulose, polyvinyl alcohol, hydroxyethyl cellulose, hydroxymethyl methacrylate, sodium carboxymethyl cellulose, calcium carboxymethyl cellulose, methyl cellulose, and natural substances, such as psyllium, maltodextrin, xanthan gum, tragacanth gum, agar, gellan gum, kayara gum, alginic acids, pectins, starch, copolymers and mixtures and blends thereof.

The term “foam”, as used herein, means and includes a hydratable material that expands upon exposure to a hydration medium, and is capable of retaining a significant fraction of formed gas by maintaining at least one pore or opening. The term “foam” thus includes, without limitation, natural gel-forming substances, either with or without further crosslinking, alone or in combination, acacia (gum Arabic), agar, agarose, alginate gels or alginic acid, including non-cross linked and cross linked alginate gums, wherein the cross linked alginate gums can be cross linked with di-or trivalent ions, amylopectin, arbinoglactan, carob bean gum, carrageenan, chondroitin sulfate, eucheuma, fucoidan, furcellaran, gelatin, gellan, guar gum, gum ghatti, gum karaya, hypnea, karaya, laminaran, locust bean gum, natural gum, pectins, polypeptides, polyamino acids, tragacanth, xanthan, and including but not limited to synthetic polymers and copolymers of either natural or synthetic origin, such as Carbopol® acidic carboxy polymer, hydrophilic poly urethanes, hydroxypropyl methyl cellulose, HYPOL® hydrophilic polyurethane polymers, polycarbophil, polyethylene oxide, poly(hydroxyalkyl methacrylate), poly(electrolyte complexes), poly(vinyl acetate) cross-linked with hydrolysable bonds, water-swellable N-vinyl lactams polysaccharides, carboxylmethyl celluloses, Cyanmer® polyacrylamides, Good-rite® polyacrylic acid, starch graft copolymers, Aqua-Keeps® acrylate polymer, ester cross linked polyglucan, polycellulosic acid, polyurea, polyether, poly(acrylic acid), polyacrylamide, or poly(2-hydroxyethyl methacrylate, methocel, and biodegradable polymers, including, without limitation, alpha-polyhydroxy acids, polyglycolide (PGA), poly(L-lactide), poly(D,L-lactide), poly(.epsilon.-caprolactone), poly(trimethylene carbonate), poly(ethylene oxide) (PEO), poly(.beta.-hydroxybutyrate) (PHB), poly(.beta.-hydroxyvalerate) (PHVA), poly(p-dioxanone) (PDS), poly(ortho esters), tyrosine-derived polycarbonates, like materials, copolymers of the above, and combinations and mixtures thereof.

The “foams” of the invention can be obtained by blowing, beating, shaking, spraying or stirring one or more of the aforementioned materials in the presence of the desired gas, such as air, while the foam material is being formed. Natural or synthetic foam stabilizing agents can also be added, such as protein, egg white, or detergents, such as SDS.

The term “wet strength”, as used herein, means and includes the tensile strength of a hydrated gastric device material or component, e.g., skin. The term “wet strength” thus also means the ability of a hydrated gastric device material to withstand grinding by the stomach structure(s).

The term “food grade”, as used herein, means and includes a material that is commonly consumed as a food (i.e. a source of nutrients) or employed as a food additive.

The term “directive”, as used herein, means the ability to move in a desired direction or be placed in a desired location.

As will be appreciated by one having ordinary skill in the art, the present invention substantially reduces or eliminates the disadvantages and shortcomings associated with prior art gastric devices. The invention generally comprises ingestible and biodegradable gastric devices that expand sufficiently in the stomach of a subject to at least partially suppress the subject's appetite. A key advantage of the gastric devices of the invention is the provision of a safe and efficient means of limiting food and, hence, caloric intake to facilitate weight reduction. The gastric devices are also readily adaptable to meet the needs of virtually any subject.

In some embodiments of the invention, the gastric devices include monitoring means for tracking and monitoring the gastric devices prior to and after administration or ingestion of the gastric devices by a subject. As discussed in detail herein, the monitoring means provides the additional advantages of readily (and non-invasively) confirming the presence of a gastric device in the stomach cavity, and determining and monitoring residence time of a gastric device in the stomach cavity and/or gastrointestinal tract, without the need for additional medical procedures, e.g., xray or ultrasound, or skilled personnel, such as a physician, and confirming elimination of a gastric device from the body.

A further advantage of the monitoring means is that it facilitates tracking of data and information associated with the manufacture, distribution, storage and use of gastric devices.

According to the invention, the gastric devices of the invention can be ingested in dry form with most ingestible fluids (e.g., water, juice, etc.) or food. The gastric devices are also robust, i.e. able to withstand grinding by the stomach muscles.

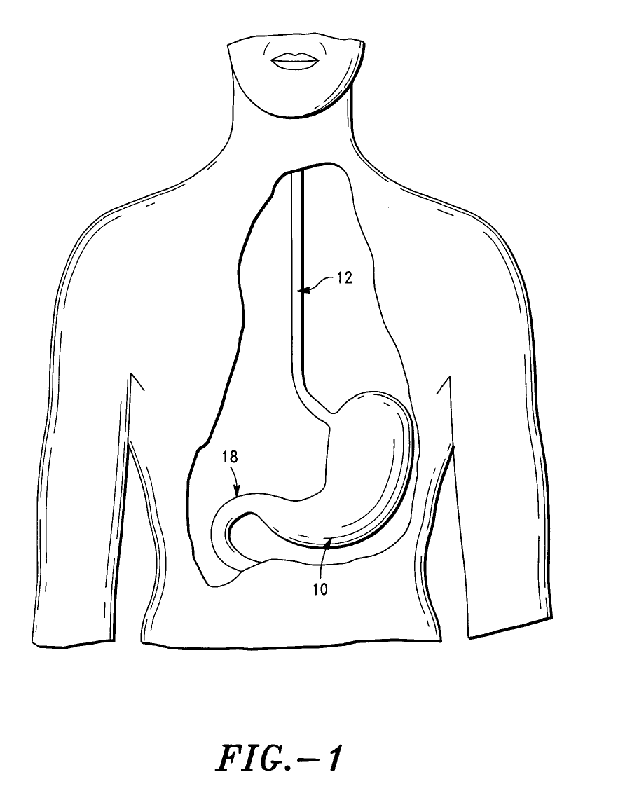

Before describing the present invention in detail, it is believed that it will be useful to briefly review the anatomy of the stomach and esophagus. Referring to FIGS. 1 and 2, the esophagus 12 is a muscular tube that carries food from the throat to the stomach 10.

The top end of the esophagus 12 is the narrowest part of the entire digestive system and is encircled by a sphincter (i.e., circular muscle) that is normally closed, but opens to allow the passage of food. There is a similar sphincter (i.e., lower esophageal sphincter) 14 where the esophagus 12 enters the stomach 10.

The stomach 10 is a sac-like organ having a fundus 11 (i.e., expanded curvature region) and a lower pyloric sphincter 16, which controls passage of food from the stomach 10 to the duodenum 18 (i.e., first part of small intestine). In addition to holding food, the stomach 10 serves as a mixer and grinder of food. The stomach 10 also secretes acid and powerful enzymes that further break down the food to form a paste or liquid.

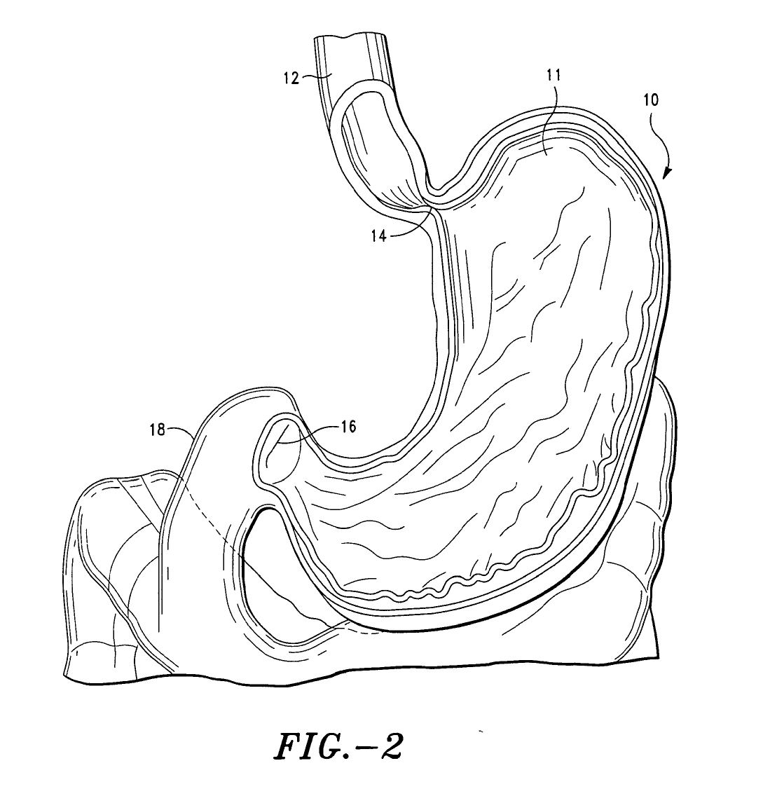

Referring now to FIGS. 3A and 3B, there is shown one embodiment of a gastric device of the invention prior to ingestion and/or hydration (FIG. 3A) and after hydration (FIG. 3B). As illustrated in FIG. 3A, prior to ingestion and hydration, the gastric device (denoted “20A”) has a substantially oblong structure 21 comprising an outer layer or skin 22 and an internal, expandable cavity 24 having a core material 23 disposed therein.

In a preferred embodiment, the core material 23 comprises a hydratable material that is adapted to effectuate expansion of the cavity 24 and, hence, gastric device structure 21 upon hydration by a hydration medium (e.g. gastric fluid), as shown in FIG. 3B.

According to the invention, the gastric device 20A (as well as all gastric devices described herein) can comprise various sizes and virtually any shape (e.g., oblong, circular, cylindrical, cube etc).

In one embodiment of the invention, the gastric device (e.g., 20A) has a substantially oblong pre-hydration shape with a maximum length in the range of approximately 2-15 cm, more preferably, in the range of approximately 3-8 cm, and a maximum width in the range of 0.4-6 cm, more preferably, in the range of approximately 0.8-2 cm.

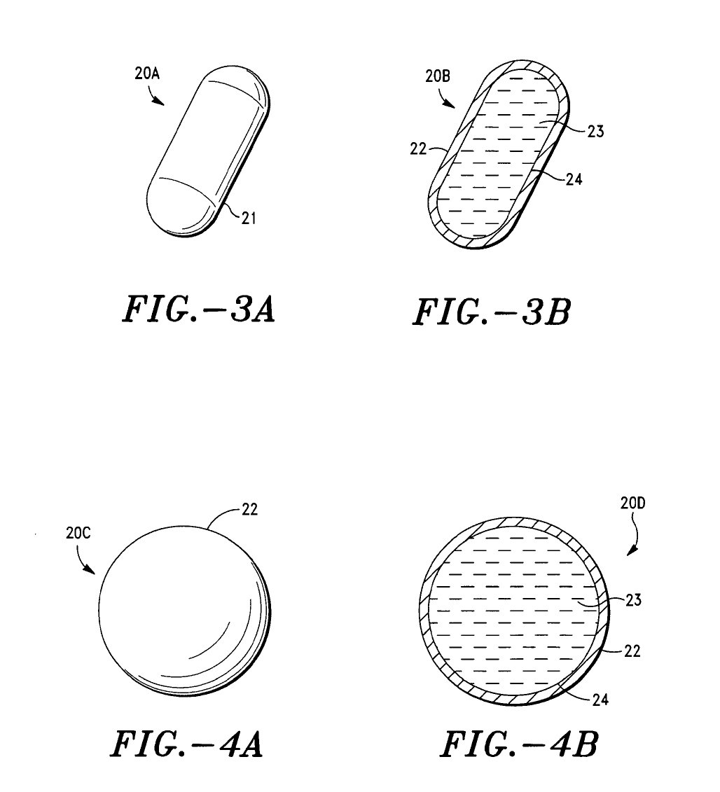

Referring now to FIGS. 4A and 4B, there is shown one embodiment of a gastric device of the invention prior to ingestion and/or hydration (FIG. 4A) and after hydration (FIG. 4B). As illustrated in FIG. 4A, prior to ingestion and hydration, the gastric device (denoted “20C”) has a substantially round or spherical structure 22 that similarly comprises an outer layer or skin 22 and an internal, expandable cavity 24 having a core material 23 disposed therein.

In some embodiments, the device 20C preferably has a pre-hydration radius in the range of approximately 0.25-6 cm.

According to the invention, the spherical gastric devices, e.g., device 20C, can also have substantially flat top and bottom surfaces (i.e. a pancake configuration).

As will be appreciated by one having ordinary skill in the art, the noted dimensions of gastric devices 20A, 20C, as well as all gastric devices of the invention, facilitate easy ingestion by a subject. The noted range(s) of expanded gastric device dimensions also facilitates control of gastric retention.

As indicated above, according to the invention, upon entry into the stomach, the gastric devices 20A, 20C absorb gastric fluid contained in the stomach (e.g., ingested fluids, hydrochloric acid, etc.) and, upon hydration of the core material(s) 23 and, hence, structures 21, 22, the gastric devices 20A, 20C expand to an enhanced volume, as shown in FIGS. 3B and 4B.

In some embodiments of the invention, upon hydration of the core material (i.e. a single core material or a mixture of core materials) 23, the core material expands to effectuate expansion of the device cavity 24 and, hence, structures 21, 22.

In some embodiments of the invention, upon hydration of the core material 23, the core material produces a gas (e.g., CO₂) that is contained within the device cavity 24. As the gas is released, the cavity 24 and, hence, gastric device structures 21, 22 expand.

As will be appreciated by one having ordinary skill in the art, the volume of the hydrated, expanded gastric devices of the invention will occupy space in the stomach cavity that is normally used for receipt of consumed substances (e.g., food, drink) and, hence, reduce the amount of food a subject will ingest before reaching the feeling of fullness.

Preferably, the gastric devices 20A, 20C (as well as all gastric devices of the invention) expand to a size that prohibits the devices from passing the lower gastric sphincter into the duodenum (or, alternatively, allows for controllable time to passage) while allowing food to pass therethrough. According to the invention, when a gastric device enters into the duodenum, the device is readily degraded and/or passed in the small and large intestines. Blockage is thus substantially reduced or eliminated.

According to the invention, the on-set and rate of hydration of the gastric devices 20A, 20C (and devices 6A and 6D, discussed below) are infinitely adjustable. In some embodiments, the gastric devices 20A, 20C have an initial delayed hydration period in the range of approximately 1 min.-30 min. to prevent the devices 20A, 20C from expanding in the esophagus. In some embodiments, the gastric devices 20A, 20C have an initial delayed hydration period in the range of approximately 5 min.-10 min.

In some embodiments of the invention, the gastric devices 20A, 20C have a partial hydration rate (i.e. approx. 50% hydration) in the range of approximately 5 min.-3 hrs. In some embodiments of the invention, the gastric devices 20A, 20C have a partial hydration rate in the range of approximately 10 min.-30 min.

In some embodiments of the invention, the gastric devices 20A, 20C reach full hydration (i.e. complete expansion) in the range of approximately 1 min.-4 hrs. after ingestion. In some embodiments of the invention, the gastric devices 20A, 20C reach full hydration within 3 hrs. after ingestion.

According to the invention, the materials (discussed below) and thickness of the outer layer or skin 22 can also be selected to achieve a desired degradation or, alternatively, passage and/or degradation rate(s) and, hence, desired residence time (e.g., 2, 6, 9, 12 or 24 hours or more) in the stomach. Upon expiration of the pre-determined residence time (i.e. degradation of the device or, alternatively, passage), the gastric device and/or device material simply passes into the intestines and through as a human fecal waste product.

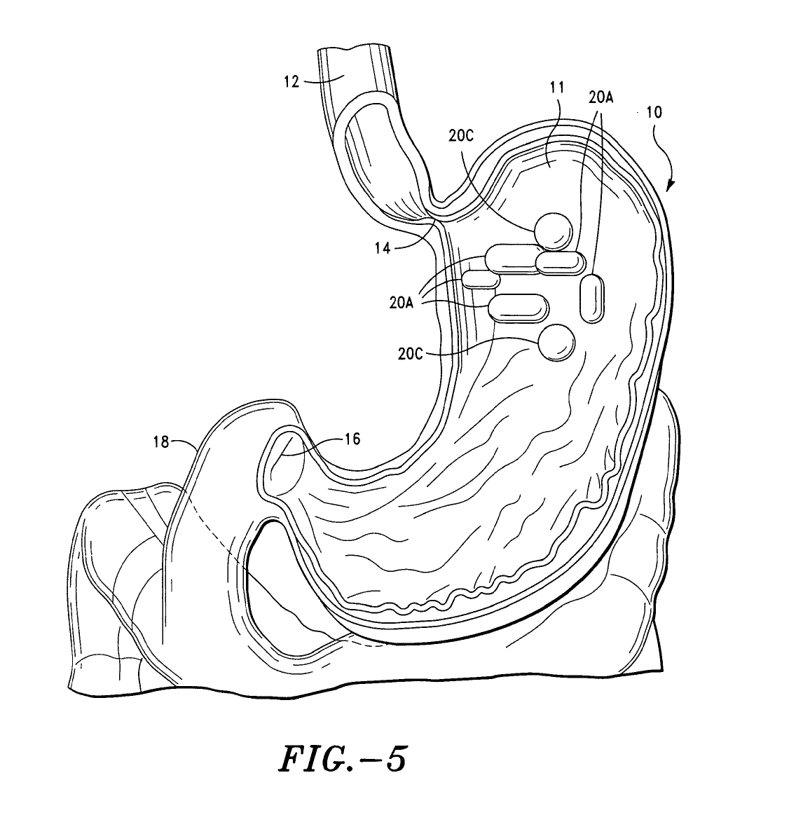

As stated above, the ingested gastric devices of the invention are also directive. As illustrated in FIG. 5, the gastric devices are thus capable of being directed to a desired point in the stomach (e.g., proximate the fundus 11).

According to the invention, the gastric devices 20A, 20C have an expansion coefficient upon hydration of approximately 3-50 fold or more. In a preferred embodiment of the invention, the gastric devices 20A, 20C have an expansion coefficient in the range of approximately 10-20. The size of the gastric devices 20A, 20C (and quantity ingested) can thus be tailored to suit a particular subject and/or weight reduction program.

In some embodiments of the invention, the outer layer or skin 22 comprises a biodegradable, preferably food grade, material, including, without limitation, natural gel-forming substances, either with or without further crosslinking, alone or in combination, acacia (gum Arabic), agar, agarose, alginate gels or alginic acid, including non-cross linked and cross linked alginate gums, wherein the cross linked alginate gums can be cross linked with di-or trivalent ions, amylopectin, arbinoglactan, carob bean gum, carrageenan, chondroitin sulfate, eucheuma, fucoidan, furcellaran, gelatin, gellan, guar gum, gum ghatti, gum karaya, hypnea, karaya, laminaran, locust bean gum, natural gum, pectins, polypeptides, polyamino acids, tragacanth, xanthan, and synthetic polymers and copolymers of either natural or synthetic origin, such as Carbopol® acidic carboxy polymer, hydrophilic poly urethanes, hydroxypropyl methyl cellulose, HYPOL® hydrophilic polyurethane polymers, polycarbophil, polyethylene oxide, poly(hydroxyalkyl methacrylate), poly(electrolyte complexes), poly(vinyl acetate) cross-linked with hydrolysable bonds, water-swellable N-vinyl lactams polysaccharides, carboxylmethyl celluloses, Cyanmer® polyacrylamides, Good-rite® polyacrylic acid, starch graft copolymers, Aqua-Keeps® acrylate polymer, ester cross linked polyglucan, polycellulosic acid, polyurea, polyether, poly(acrylic acid), polyacrylamide, or poly(2-hydroxyethyl methacrylate, methocel, and biodegradable polymers, including, without limitation, alpha-polyhydroxy acids, polyglycolide (PGA), poly(L-lactide), poly(D,L-lactide), poly(.epsilon.-caprolactone), poly(trimethylene carbonate), poly(ethylene oxide) (PEO), poly(.beta.-hydroxybutyrate) (PHB), poly(.beta.-hydroxyvalerate) (PHVA), poly(p-dioxanone) (PDS), poly(ortho esters), tyrosine-derived polycarbonates, like materials, copolymers of the above, and combinations and mixtures thereof. Preferred gelling materials for the outer layer 32 are digestible natural substances, such as food grade agar and carrageenan.

In some embodiments of the invention, the skin 22 also includes reinforcing fibers to enhance the structural integrity of the gastric device, e.g., device 20A and/or 20C. Suitable reinforcing fibers include, without limitation, soluble fibers and insoluble fibers.

In some embodiments of the invention, the soluble reinforcing fibers comprise polysaccharide gums, including, without limitation, carboxymethyl cellulose, methocel, carrageenan, guar gum, algimate gels, pectins, xanthan, gum Arabic (acacia), gum tragacanth, karaya, agar, gellan, and like materials, and biodegradable polymers, including, without limitation, alpha-polyhydroxy acids, polyglycolide (PGA), poly(L-lactide), poly(D,L-lactide), poly(.epsilon.-caprolactone), poly(trimethylene carbonate), poly(ethylene oxide) (PEO), poly(.beta.hydroxybutyrate) (PHB), poly(.beta.-hydroxyvalerate) (PHVA), poly(p-dioxanone) (PDS), poly(ortho esters), tyrosine-derived polycarbonates, and polypeptides and copolymers of the above.

Additional biodegradable polymers, which can be employed within the scope of the present invention, are set forth in U.S. Pat. Nos. 6,858,222, 6,272,258, 6,596,296 and 4,147,779; the disclosures of which are expressly incorporated by reference herein.

In some embodiments of the invention, the insoluble reinforcing fibers comprise insoluble polysaccharides, including, without limitation, cellulose and polymers of phenylpropane molecules (e.g., lignin), bast fibers, Kozo or Gampi. Additional fiber sources include grasses, kenaf, bagasse, jute, hemp and flax.

In some embodiments, the skin 22 is sealed with a gum material, such as high gel strength agar. As will be appreciated by one having ordinary skill in the art, the high gel strength agar provides additional wear resistance.

In some embodiments of the invention, the core material 23 preferably comprises a gel-forming material, including, without limitation, natural gel-forming substances, either with or without further crosslinking, alone or in combination, acacia (gum Arabic), agar, agarose, alginate gels or alginic acid, including non-cross linked and cross linked alginate gums, wherein the cross linked alginate gums can be cross linked with di-or trivalent ions, amylopectin, arbinoglactan, carob bean gum, carrageenan, chondroitin sulfate, eucheuma, fucoidan, furcellaran, gelatin, gellan, guar gum, gum ghatti, gum karaya, hypnea, karaya, laminaran, locust bean gum, natural gum, pectins, polypeptides, polyamino acids, tragacanth, xanthan, and synthetic polymers and copolymers of either natural or synthetic origin, such as Carbopol® acidic carboxy polymer, hydrophilic poly urethanes, hydroxypropyl methyl cellulose, HYPOL® hydrophilic polyurethane polymers, polycarbophil, polyethylene oxide, poly(hydroxyalkyl methacrylate), poly(electrolyte complexes), poly(vinyl acetate) cross-linked with hydrolysable bonds, water-swellable N-vinyl lactams polysaccharides, carboxylmethyl celluloses, Cyanmer® polyacrylamides, Good-rite® polyacrylic acid, starch graft copolymers, Aqua-Keeps® acrylate polymer, ester cross linked polyglucan, polycellulosic acid, polyurea, polyether, poly(acrylic acid), polyacrylamide, or poly(2-hydroxyethyl methacrylate, methocel, and biodegradable polymers, including, without limitation, alpha-polyhydroxy acids, polyglycolide (PGA), poly(L-lactide), poly(D,L-lactide), poly(.epsilon.-caprolactone), poly(trimethylene carbonate), poly(ethylene oxide) (PEO), poly(.beta.-hydroxybutyrate) (PHB), poly(beta.-hydroxyvalerate) (PHVA), poly(p-dioxanone) (PDS), poly(ortho esters), tyrosine-derived polycarbonates, like materials, copolymers of the above, and combinations and mixtures thereof.

In some embodiments of the invention, the gel forming material comprises a gas forming material.

In some embodiments of the invention, the core material 23 comprises or includes an acid and a base, which, when hydrated, form a gas. By way of example, it is well known in the art that sodium bicarbonate, a preferred material, releases CO₂ when hydrated in the presence of an acid.

Suitable bases include, without limitation, sodium bicarbonate, sodium carbonate, hydrogen carbonates or carbonates of alkali metals or organic cations, such as ammonium derivatives and sodium azide, and combinations and mixtures thereof.

Suitable acids, include, without limitation, citric acid, ascorbic acid, ascorbic acid, citric acid, fumaric acid, oxalic acid, succinic acid, tartaric acid, maleic acid and inorganic acids, such as phosphoric acid/dihydrogen phosphate mixtures, like acids and mixtures thereof.

According to the invention, solid particles of the aforementioned bases and acids can also be coated with a thin protective layer, such as a hydrophilic, gel-forming material, to maintain their functional integrity during storage.

In some embodiments of the invention, the gel forming material comprises a foam.

According to the invention, the core material 23 and/or skin 22 can also include a viscosity building material, including, without limitation, water soluble polymers, sodium and calcium polyacrylic acid, polyacrylic acid, polymethacrylic acid, polymethylvinylether co-maleic anhydride, polyvinylpyrrolidone, polyethylene oxide, hydroxypropyl cellulose, hydroxypropyl methyl cellulose, polyvinyl alcohol, hydroxyethyl cellulose, hydroxymethyl methacrylate, sodium carboxymethyl cellulose, calcium carboxymethyl cellulose, methyl cellulose, and natural substances, such as psyllium, maltodextrin, xanthan gum, tragacanth gum, agar, gellan gum, kayara gum, alginic acids, pectins, starch, copolymers and mixtures and blends thereof.

The gastric devices 20A, 20C can also include enzymes that aid in the degradation of the skin 22 following ingestion of the device.

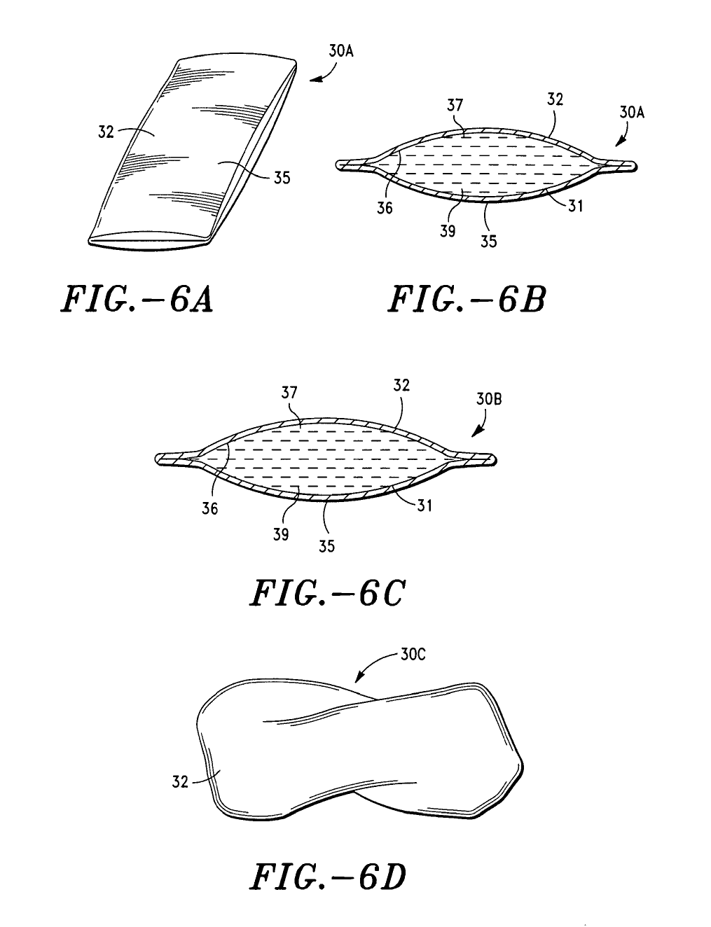

Referring now to FIGS. 6A and 6B, there is shown another embodiment of a gastric device of the invention. As illustrated in FIG. 6B, the gastric device 30A generally comprises a “pouch” that includes an outer layer or skin 32 having a sealed cavity 37 with a hydratable core material 39 disposed therein.

According to the invention, the skin 32 can be formed from various food grade materials or “papers” that are degradable by gastric fluid. In some embodiments of the invention, the skin 32 comprises at least one of the aforementioned gel-forming materials. In some embodiments, the skin 32 is also preferably reinforced with fibers, more preferably, one or more of the aforementioned insoluble fibers.

In some embodiments of the invention, the skin 32 additionally includes a coating on the outer surface 35 to delay or modify the hydration rate of the skin 32 and/or core material 39 or facilitate easy ingestion of the device. Such coatings can thus, include, without limitation, proteinaceous materials, such as gelatin to enhance digestion, and high gel-strength materials, such as agar to facilitate longer residence times.

According to the invention, the thickness of the skin 32 and/or material employed to form same can similarly be selected to control the on-set and rate of hydration of the gastric device 30A.

In some embodiments of the invention, the hydratable core material comprises at least one of the aforementioned gel forming materials.

In one embodiment of the invention, at least one of the aforementioned materials that form a gas when hydrated (e.g., base/acid, gel-forming agent, etc.) is disposed in the inner cavity 31 of the gastric device 30A.

According to the invention, a viscosity building material and/or any of the aforementioned supplemental materials can also be disposed in the inner cavity 31.

Referring now to FIG. 6C, upon hydration of the core material 39 the gastric device 30A expands to an enhanced volume, forming device 30B.

In some embodiments of the invention, upon hydration of the core material 39, the core material 39 expands to effectuate expansion of the device cavity 31 and, hence, device 30A.

In some embodiments of the invention, wherein the core material 39 comprises a gas producing material (or materials), upon hydration of the core material 39, the core material (or materials) 39 produces a gas (e.g., CO₂) that inflates and expands the device cavity 31 and, hence, gastric device 30A.

According to the invention, the gastric device 30A can similarly comprise a multitude of sizes. The formed (or post-hydration) device 30B can also be formed from various shapes, such as a rolled or twisted pre-hydration member or device, such as shown in FIG. 6D and denoted device 30C, and into various shapes.

According to the invention, the gastric device 30A (and/or gastric device 30C) can have dimensions similar to devices 20A and 20B, discussed above. The gastric device 30A (and/or 30C) also has hydration and degradation characteristics similar to gastric devices 20A, 20B.

The gastric device 30A (and/or gastric device 30C) also has an adjustable residence time (e.g., hours, days, weeks, etc.) in the stomach cavity. In a preferred embodiment, the residence time of the gastric device 30A (and/or 30C) in the stomach cavity is in the range of approximately 1 hr.-1 year, more preferably, in the range of approximately 3 hours to three weeks.

Further details of the aforementioned gastric devices are set forth in U.S. Pat. No. 7,674,396 and Co-pending U.S. application Ser. No. 11/270,723; which are incorporated by reference herein in their entirety.

In some embodiments of the invention, the gastric devices include means for monitoring the devices prior to and after administration or ingestion by a subject. In some embodiments, the monitoring means comprises a biocompatible radio frequency identification (RFID) tag.

As is well known in the art, a typical RFID tag includes a microchip and an associated antenna, which are disposed on a substrate. The tag is often attached to a product or product container to identify the product and/or provide other relevant information associated with the product, e.g., date of manufacture.

The antenna typically comprises a material that can be caused to sympathetically resonate by a field attuned to a particular frequency, e.g., in the radio frequency range, and is adapted to receive remote queries (or signals) from an interrogation device, e.g., scanner or reader, and transmit signals representing stored data, e.g., identification number of attached product, back to the interrogation device.

Illustrative are the RFID tags disclosed in U.S. Pat. Nos. 5,682,143 and 6,894,614; which are incorporated by reference herein.

A RFID system thus provides effective means of identifying, monitoring, and controlling products, devices, materials, etc. in a closed loop process. In a factory environment, the tags are employed as the transport mechanism between “islands of automation,” providing a record of each process that can be acted upon immediately or downloaded later for analysis.

Biocompatible RFID tags have also been recently developed for implantation in and ingestion into a living body to, for example, monitor administered pharmaceuticals. Illustrative are the RFID tags disclosed in U.S. Pat. Nos. 5,850,196, 5,874,099 and 7,782,189.

A drawback associated with ingestible RFID tags, such as the tag disclosed in U.S. Pat. No. 7,782,189, is that the tags are not biodegradable and can, and in many instances will, become lodged in the gastrointestinal tract during passage therethrough.

As discussed in detail herein, the RFID tags of the invention are encapsulated in a biocompatible material and formed in a shape that substantially reduces the probability of the RFID tag becoming lodged in the gastrointestinal tract during passage therethrough.

Referring now to FIG. 7, there is shown a prior art passive RFID tag 40, which is employed in one embodiment of the invention. As illustrated in FIG. 7, the tag 40 includes a microchip 42 and an associated antenna 44, which are disposed on a substrate 46.

In a preferred embodiment of the invention, the tag 40 is encapsulated with a biocompatible material to aid in the passage of the tag 40 though the gastrointestinal tract. In some embodiments of the invention, the encapsulation material comprises a biocompatible polymeric material. Suitable polymeric materials include, without limitation, of poly(urethanes), poly(siloxanes), poly(methyl methacrylate), poly(vinyl alcohol) for hydrophilicity and strength, poly(ethylene), poly(vinyl pyrrolidone, poly(2-hydroxy ethyl methacrylate), poly(N-vinyl pyrrolidone), poly(methyl methacrylate), poly(vinyl alcohol), poly(acrylic acid), polyacrylamide, poly(ethylene-co-vinyl acetate), poly(ethylene glycol), poly(methacrylic acid), polylactides (PLA), polyglycolides (PGA), poly(lactide-co-glycolides) (PLGA), polyanhydrides, polyorthoesters, like materials and combinations thereof.

In some embodiments of the invention, the tag 40 is encapsulated with one of the aforementioned gel-forming materials.

In some embodiments, the tag 40 is encapsulated with one of the aforementioned food grade materials.

According to the invention, the tag 40 can be encapsulated with any of the foregoing materials to form any desired shape, e.g., oblong, spherical, etc. to further aid in the passage of the tag 40 though the gastrointestinal tract. In some embodiments, as shown in FIG. 8, the encapsulant 48 (and encased tag 40) have a substantially round or spherically shaped structure 50.

In some embodiments of the invention, the encapsulated RFID tags of the invention have a length no greater than approximately 5 mm-10 mm and a width no greater than 5 mm-10 mm, and if spherical in shape, a diameter no greater than approximately 5 mm-10 mm.

In some embodiments of the invention, the spherically shaped encapsulated tags have a diameter no greater than 2 mm. As will readily be appreciated by one have ordinary skill in the art, the encapsulated RFID tags of the invention thus have dimensions similar to a grain of sand.

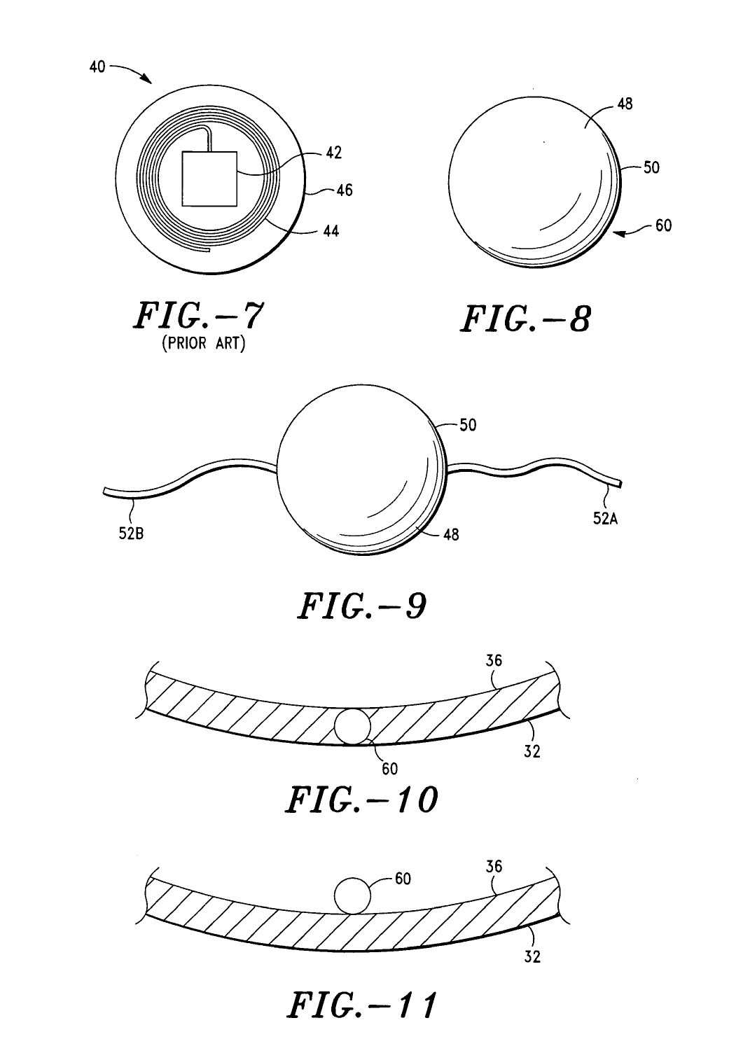

Referring now to FIG. 9, in some embodiments of the invention, the encapsulated structure, in this instance structure 50, includes at least one, more preferably, a plurality of extending stringers 52a, 52b, that are positioned and configured to further reduce the probability of the RFID tag 40 becoming lodged in the gastrointestinal tract during passage therethrough.

According to the invention, the stringers, e.g., 52a, 52b, can comprise any length. In some embodiments, the stringers preferably have a length in the range of approximately 0.5-5 cm. In some embodiments, the stringers preferably have a length in the range of approximately 1-3 cm.

According to the invention, the unique encapsulated RFID tags of the invention can be employed with virtually any ingested gastric retention device, including gastric devices 20A, 20C, 30A and 30C, discussed above.

The encapsulated RFID tags can also be disposed at various positions in or on a gastric device, such as in or on the outer layer or skin, in a device cavity and/or within the core material matrix.

Referring now to FIGS. 10 and 11, there are shown partial sectional views of gastric device 30A having an encapsulated RFID tag 60 associated therewith. Referring first to FIG. 10, in some embodiments of the invention, the tag 60 is disposed in the skin 32 of the device 30A.

As illustrated in FIG. 11, in some embodiments, the tag 60 is disposed in the cavity 37 of the device 30A. According to the invention, the tag 60 can be disposed within the core material matrix 39 or, as shown in FIG. 11, disposed proximate or attached to the inner surface 36 of the skin 32 using, for example, a biocompatible adhesive.

According to the invention, various data and information associated with a gastric device, such as devices 20A, 20C and 30A, can be stored in the tag microchip 42. In some embodiments of the invention, the accessible data stored on the tag microchip 42 includes at least a unique device identification number. In some embodiments, the identification number preferably comprises a non-repeated number and a unique code to identify the device.

According to the invention, additional data associated with a gastric device (and unique identification number) that can be stored in the tag microchip 42, includes, without limitation, the date of manufacture, manufacturing lot, expiration date, manufacturing location and other relevant information typically associated with medical device manufacturing records under Good Manufacturing Practices.

Preferably, the additional data is contained in a database maintained by one or more designated parties, such as the manufacturer, and accessible by various means during the use of the gastric device(s) or at an appropriate time when such data would be useful to the patient, care-giver or manufacturer.

According to the invention, some or all of the aforementioned data could be encoded within the microchip 42 at the time of manufacture in order to make the information available separate from, and without need to access, the database.

Additional or supplemental data and information that can be stored in the tag microchip 42 include, without limitation, data and information associated with a subject, e.g., name, age, weight, etc., the day and time of administration, day and time of movement from the stomach, retention time within the stomach, retention time within the intestine, and retention time within the body before elimination.

Preferably, the noted supplemental data is similarly retained in a database, which is similarly accessible by various means during the use of the gastric device or at an appropriate time when such data would be useful to the patient or care-giver.

According to the invention, the noted data and information can be stored in the microchip 42 prior to encapsulation and accessed by a conventional associated interrogator or RFID reader, forming an RFID system of the invention. Suitable readers include, without limitation, the Enterprise 9900+ and ALR-9650 Gen 2 readers distributed by Alien Corp., the XCRF-860 Gen 2 reader distributed by Invengo Corp., and the 5500 reader distributed by Datalogic.

In a preferred embodiment of the invention, the RFID reader (when in communication with a tag and, hence gastric device associated therewith) is adapted to at least (i) localize a gastric device within defined parts of the body, such as the stomach, intestinal tract and colon, (ii) identify the gastric device by unique ID number prior to ingestion and during the retention in the stomach cavity and passage through the patient, and (iii) record the appropriate data and information (as indicated above) in a database within the RFID reader.

In some embodiments, the RFID reader is further adapted to provide means of communicating the information tied to the unique ID number to the patient, care-giver and manufacturer through appropriate means. According to the invention, suitable communication means include, without limitation, a display screen on the reader, printout through appropriate printer linkage and uploading the database via various conventional techniques, such as internet access.

The RFID system of the invention can thus be employed to readily (and non-invasively) confirm the presence of a gastric device or a plurality of gastric devices in the stomach cavity, and/or determine and monitor residence time of one or more gastric devices in the stomach cavity and/or gastrointestinal tract, and confirm elimination of each gastric device from the body, without the need for additional medical procedures, e.g., xray or ultrasound, or skilled personnel.

To reduce the probability of tag interference resulting from energizing closely grouped gastric devices (having RFID tags associated therewith), i.e. simultaneous signal transmissions that are difficult to differentiate, in some embodiments of the invention, anti-collision protocols and algorithms, i.e. binary-tree and ALOHA, are employed. Suitable anti-collision mechanisms are described in Zhou, et al., “Evaluating and Optimizing Power Consumption of Anti-Collision Protocols for Applications in RFID systems,” Proceedings of the International Symposium on Low Power Electronics and Design, pp. 357-362 (2004); which is incorporated by reference herein.

In some embodiments of the invention, each RFID microchip and tag circuit is adapted to receive an assigned modulated RF signal from a RFID interrogator or reader and provide a unique modulated signal in response thereto. Such a system is disclosed in U.S. Pat. Pub. No. 2010/0182129; which is also incorporated by reference herein.

According to the invention, the unique encapsulated RFID tags of the invention can also be employed with virtually any ingested device or system (e.g., Pillcam™) and/or pharmaceutical or medication (e.g., tablets and pills) to monitor the system and/or pharmaceutical prior to and/or after administration by a subject or patient.

It is to be understood that although the monitoring means of the invention comprises a system utilizing radio frequency tags, the simplicity of the monitoring means allows for the use of acoustical frequency tags, as well as tags that reflect or resonate in other frequencies.

It is further to be understood that although the monitoring means described herein comprises a passive tag, the invention is not limited to passive RFID tags. According to the invention, the monitoring means can also comprise active tags.

The monitoring means of the invention can also comprise one or more field programmable RFID tags. As is well known in the art, field-programmable tags typically include two forms of memory; a programmable chip and a form of read-only memory containing a unique serial number imposed at the factory.

As will be appreciated by one having ordinary skill in the art, the present invention provides numerous advantages. Among the advantages are the following:
The provision of a method and system for treatment of eating disorders that is low-risk, unobtrusive and noninvasive;
The provision of ingestible gastric devices that minimize or eliminate the possibility of esophageal expansion during ingestion;
The provision of ingestible, inflatable gastric devices that substantially reduce or eliminate passage through the pyloric sphincter when inflated;
The provision of ingestible, inflatable gastric devices having a controlled rate of inflation and, hence, expansion;
The provision of ingestible gastric device having a controlled rate of degradation;
The provision of ingestible, inflatable gastric devices that include means for monitoring the gastric devices prior to and/or after administration by a subject; and
The provision of unique monitoring means that can be employed with virtually any ingested device or system (e.g., Pillcam™) and/or pharmaceutical or medication (e.g., tablets and pills) to monitor the system and/or pharmaceutical prior to and/or after administration by a subject or patient.

Without departing from the spirit and scope of this invention, one of ordinary skill can make various changes and modifications to the invention to adapt it to various usages and conditions. As such, these changes and modifications are properly, equitably, and intended to be, within the full range of equivalence of the following claims.

What is claimed is:

1. An ingestible gastric device, comprising:
a formed body, said body being adapted to expand upon exposure to a hydration medium; and
monitoring means for monitoring the gastric device.

2. The gastric device of claim 1, wherein said body includes an outer layer adapted to receive a core material therein.

3. The gastric device of claim 1, wherein said monitoring means comprises a passive RFID tag.

4. The gastric device of claim 3, wherein said RFID tag is encapsulated in a material selected from the group consisting of poly(urethanes), poly(siloxanes), poly(methyl methacrylate), poly(vinyl alcohol) for hydrophilicity and strength, poly(ethylene), poly(vinyl pyrrolidone, poly(2-hydroxy ethyl methacrylate), poly(N-vinyl pyrrolidone), poly(methyl methacrylate), poly(vinyl alcohol), poly(acrylic acid), polyacrylamide, poly(ethylene-co-vinyl acetate), poly(ethylene glycol), poly(methacrylic acid), polylactides (PLA), polyglycolides (PGA), poly(lactide-co-glycolides) (PLGA), polyanhydrides, polyorthoesters, and mixtures and combinations thereof.

5. A food grade ingestible gastric device, comprising:
a flexible pouch formed from a food grade biodegradable film, said pouch having a first volume that allows normal ingestion of said gastric device into a subject's stomach, said pouch enclosing a sealed cavity having a core material disposed therein, said biodegradable film comprising a first gel-forming material selected from the group consisting of acacia (gum Arabic), agar, agarose, alginate gels, alginic acid, alginate gums, amylopectin, arabinogalactan, carob bean gum, carrageenan, chondroitin sulfate, eucheuma, fucoidan, furcellaran, gelatin, gellan, guar gum, gum ghatti, gum karaya, hypnea, laminaran, locust bean gum, natural gum, pectins, starches, polypeptides, polyamino acids, tragacanth, xanthan, psyllium, maltodextrin, and mixtures and combinations thereof,
said core material comprising a hydratable material that is adapted to effectuate expansion of said sealed cavity upon hydration of said hydratable material by a gastric fluid, whereby upon a first hydration of said degradable film and, thereby, said hydratable material, by a first gastric fluid, said gastric device expands to a second volume greater than said first volume, said second volume being sufficient to preclude passage of said gastric device into said subject's duodenum while still allowing food to pass therethrough,
said gastric device being adapted to commence said first hydration after contact with said first gastric fluid for a period of time in the range of approximately 5-30 min,
said gastric device being adapted to sufficiently degrade in said subject's stomach after a first period of time to allow passage of said gastric device into said subject's duodenum;
said gastric device further including monitoring means for non-invasively monitoring the gastric device while disposed in a subject's gastrointestinal tract.

6. The gastric device of claim 5, wherein said monitoring means comprises a passive RFID tag.

7. The gastric device of claim 6, wherein said RFID tag is encapsulated in a material selected from the group consisting of poly(urethanes), poly(siloxanes), poly(methyl methacrylate), poly(vinyl alcohol) for hydrophilicity and strength, poly(ethylene), poly(vinyl pyrrolidone, poly(2-hydroxy ethyl methacrylate), poly(N-vinyl pyrrolidone), poly(methyl methacrylate), poly(vinyl alcohol), poly(acrylic acid), polyacrylamide, poly(ethylene-co-vinyl acetate), poly(ethylene glycol), poly(methacrylic acid), polylactides (PLA), polyglycolides (PGA), poly(lactide-co-glycolides) (PLGA), polyanhydrides, polyorthoesters, and mixtures and combinations thereof.

8. The gastric device of claim 5, wherein said hydratable material comprises a second gel-forming material selected from the group consisting of acacia (gum Arabic), agar, agarose, alginate gels, alginic acid, alginate gums, amylopectin, arabinogalactan, carob bean gum, carrageenan, chondroitin sulfate, eucheuma, fucoidan, furcellaran, gelatin, gellan, guar gum, gum ghatti, gum karaya, hypnea, laminaran, locust bean gum, natural gum, pectins, starches, polypeptides, polyamino acids, tragacanth, xanthan, psyllium, maltodextrin, and mixtures and combinations thereof.

9. The gastric device of claim 8, wherein said first and second gel-forming materials are selected from the group consisting of acacia (gum Arabic), agar, agarose, alginate gels, alginic acid, alginate gums, amylopectin, arbinoglactan, carob bean gum, carrageenan, chondroitin sulfate, eucheuma, fucoidan, furcellaran, gelatin, gellan, guar gum, gum ghatti, gum karaya, hypnea, karaya, laminaran, locust bean gum, natural gum, pectins, starches, polypeptides, polyamino acids, tragacanth, xanthan, psyllium, maltodextrin, Carbopol® acidic carboxy polymer, hydrophilic poly urethanes, hydroxypropyl methyl cellulose. HYPOL® hydrophilic polyurethane polymers, polycarbophil, polymethylvinylether co-maleic anhydride, polyvinylpyrrolidone, polyethylene oxide, poly(hydroxyalkyl methacrylate), polymethacrylic acid, poly(electrolyte complexes), poly(vinyl acetate) cross-linked with hydrolysable bonds, polyvinyl alcohol, water-swellable N-vinyl lactams polysaccharides, hydroxypropyl cellulose, carboxylmethyl celluloses, hydroxyethyl cellulose, methyl cellulose, polyvinyl alcohol, hydroxymethyl methacrylate, Cyanmer® polyacrylamides, Good-rite® polyacrylic acid, starch graft copolymers, Aqua-Keeps® acrylate polymer, ester cross linked polyglucan, polycellulosic acid, polyurea, polyether, poly(acrylic acid), polyacrylamide, or poly(2-hydroxyethyl methacrylate, hydroxymethyl methacrylate, methocel, alpha-polyhydroxy acids, polyglycolide (PGA), poly(L-lactide), poly(D,L-lactide), poly(.epsilon.-caprolactone), poly(trimethylene carbonate), poly(ethylene oxide) (PEO), poly(.beta.-hydroxybutyrate) (PHB), poly(.beta.-hydroxyvalerate) (PHVA), poly(p-dioxanone) (PDS), poly(ortho esters), tyrosine-derived polycarbonates, and mixtures and combinations thereof.

10. The gastric device of claim 9, wherein said second gel-forming material comprises a gas producing material.

11. The gastric device of claim 9, wherein said second gel-forming material comprises a foam.

12. The gastric device of claim 5, wherein said core material comprises a polymeric foam, said polymeric foam comprising synthetic polymers and copolymers selected from the group consisting of Carbopol® acidic carboxy polymer, hydrophilic poly urethane, hydroxypropyl methyl cellulose, HYPOL® hydrophilic polyurethane polymers, polycarbophil, polyethylene oxide, poly(hydroxyalkyl methacrylate), poly(electrolyte complexes), poly(vinyl acetate) cross-linked with hydrolysable bonds, water-swellable N-vinyl lactams polysaccharides, carboxylmethyl celluloses, Cyanmer® polyacrylamides, Good-rite® polyacrylic acid, starch graft copolymers, Aqua-Keeps® acrylate polymer, ester cross linked polyglucan, polycellulosic acid, polyurea, polyether, poly(acrylic acid), polyacrylamide, and mixtures and combinations thereof.

13. The gastric device of claim 12, wherein said polymeric foam comprises a polymer selected from the group consisting of alpha-polyhydroxy acids, polyglycolide (PGA), poly(L-lactide), poly(D,L-lactide), poly(.epsilon.-caprolactone), poly(trimethylene carbonate), poly(ethylene oxide) (PEO), poly(.beta.-hydroxybutyrate) (PHB), poly(.beta.-hydroxyvalerate) (PHVA), poly(p-dioxanone) (PDS), poly(ortho esters), tyrosine-derived polycarbonates, and mixtures and combinations thereof.

14. The gastric device of claim 5, wherein said biodegradable film further includes reinforcing fibers.

15. The gastric device of claim 14, wherein said reinforcing fibers comprise soluble reinforcing fibers selected from the group consisting of polysaccharide gums, including, without limitation, carboxymethyl cellulose, methocel, carrageenan, guar gum, algimate gels, pectins, xanthan, gum Arabic (acacia), gum tragacanth, karaya, agar, gellan, alpha-polyhydroxy acids, polyglycolide (PGA), poly(L-lactide), poly(D,L-lactide), poly(.epsilon.-caprolactone), poly(trimethylene carbonate), poly(ethylene oxide) (PEO), poly(.beta.hydroxybutyrate) (PHB), poly(.beta.-hydroxyvalerate) (PHVA), poly(p-dioxanone) (PDS), poly(ortho esters), tyrosine-derived polycarbonates, and mixtures and combinations thereof.

16. The gastric device of claim 14, wherein said reinforcing fibers comprise insoluble reinforcing fibers selected from the group consisting of insoluble polysaccharides, cellulose, phenyl propane molecules polymers, lignin, bast fibers, Kozo, Gampi, grasses, kenaf, bagasse, jute, hemp, flax, and mixtures and combinations thereof.

17. A method for monitoring a gastric device, comprising the steps of:
providing a gastric device having a formed body, said body being adapted to expand upon exposure to a hydration medium, and monitoring means for monitoring the gastric device;
administering said gastric device to a subject; and
externally interrogating said monitoring means to ascertain first data associated with the gastric device.

18. The method of claim 17, wherein said monitoring means comprises a passive RFID tag.

19. The method of claim 18, wherein said RFID tag is encapsulated in a material selected from the group consisting of poly(urethanes), poly(siloxanes), poly(methyl methacrylate), poly(vinyl alcohol) for hydrophilicity and strength, poly(ethylene), poly(vinyl pyrrolidone, poly(2-hydroxy ethyl methacrylate), poly(N-vinyl pyrrolidone), poly(methyl methacrylate), poly(vinyl alcohol), poly(acrylic acid), polyacrylamide, poly(ethylene-co-vinyl acetate), poly(ethylene glycol), poly(methacrylic acid), polylactides (PLA), polyglycolides (PGA), poly(lactide-co-glycolides) (PLGA), polyanhydrides, polyorthoesters, and mixtures and combinations thereof .

20. The method of claim 17, wherein said body includes an outer layer adapted to receive a core material therein.

21. The method of claim 20, wherein said outer layer encases a cavity having a core material disposed therein.

22. The method of claim 21, wherein said outer layer and core material comprise a gel-forming material selected from the group consisting of acacia (gum Arabic), agar, agarose, alginate gels, alginic acid, alginate gums, amylopectin, arbinoglactan, carob bean gum, carrageenan, chondroitin sulfate, eucheuma, fucoidan, furcellaran, gelatin, gellan, guar gum, gum ghatti, gum karaya, hypnea, karaya, laminaran, locust bean gum, natural gum, pectins, starches, polypeptides, polyamino acids, tragacanth, xanthan, psyllium, maltodextrin, Carbopol® acidic carboxy polymer, hydrophilic poly urethanes, hydroxypropyl methyl cellulose, HYPOL® hydrophilic polyurethane polymers, polycarbophil, polymethylvinylether co-maleic anhydride, polyvinylpyrrolidone, polyethylene oxide, poly(hydroxyalkyl methacrylate), polymethacrylic acid, poly(electrolyte complexes), poly(vinyl acetate) cross-linked with hydrolysable bonds, polyvinyl alcohol, water-swellable N-vinyl lactams polysaccharides, hydroxypropyl cellulose, carboxylmethyl celluloses, hydroxyethyl cellulose, methyl cellulose, polyvinyl alcohol, hydroxymethyl methacrylate, Cyanmer® polyacrylamides, Good-rite® polyacrylic acid, starch graft copolymers, Aqua-Keeps® acrylate polymer, ester cross linked polyglucan, polycellulosic acid, polyurea, polyether, poly(acrylic acid), polyacrylamide, or poly(2-hydroxyethyl methacrylate, hydroxymethyl methacrylate, methocel, alpha-polyhydroxy acids, polyglycolide (PGA), poly(L-lactide), poly(D,L-lactide), poly(.epsilon.-caprolactone), poly(trimethylene carbonate), poly(ethylene oxide) (PEO), poly(.beta.-hydroxybutyrate) (MB), poly(.beta.-hydroxyvalerate) (PHVA), poly(p-dioxanone) (PDS), poly(ortho esters), tyrosine-derived polycarbonates, and mixtures and combinations thereof.
